(19) United States
(12) Patent Application Publication
(10) Pub. No.: US 2023/0203072 A1
(43) Pub. Date: Jun. 29, 2023

(54) AIR-STABLE IMIDO ALKYLIDENE COMPLEXES AND USE THEREOF IN OLEFIN METATHESIS REACTIONS

(71) Applicant: Verbio Vereinigte BioEnergie AG, Zörbig (DE)

(72) Inventor: Henrik Gulyás, Budapest (HU)

(21) Appl. No.: 17/999,197

(22) PCT Filed: May 27, 2021
(86) PCT No.: PCT/EP2020/064232

§ 371 Date: Nov. 17, 2022

(30) Foreign Application Priority Data
May 27, 2020 (EP) 20176815.7

(51) Int. Cl. C07F 11/00

(52) U.S. Cl. CPC C07F 11/00

(57) ABSTRACT

The invention relates to Schrock-alkylidene complexes comprising a phenanthroline ligand. The complexes may dissociate when subjected to a solvent and release the complex being catalytically active in an olefinic metathesis reaction without needing a Lewis acid such as zinc chloride for removing the ligand. Thus, the phenanthroline complexes are autoactivating.

### FIELD OF THE INVENTION

The invention relates to 18-electron molybdenum or tungsten alkylidene complexes formed by a 14-electron molybdenum or tungsten alkylidene complex and a 1,10-phenanthroline as a neutral bidentate ligand. The invention further relates to a method of making the 18-electron catalyst, to a system comprising the 18-electron complex and the 14-electron complex, to a method of making the 14-electron complex from the 18-electron complex, and to a method of performing an olefin metathesis reaction using the complexes.

### BACKGROUND OF THE INVENTION

Olefin metathesis reactions catalyzed by transition metal catalysts such as molybdenum or tungsten alkylidene catalysts—so-called Schrock catalysts—are among the most important reactions of organic synthetic chemistry. A valuable type of known catalysts is the group of metal imido alkylidene complexes. The efficacy thereof depends on the type of metal, alkylidene group and ligands. While such catalysts have proved effective, they frequently lack stability in air which makes them more difficult to handle and often restricts their usefulness.

For improving air-stability, WO 2012/116695 suggests stabilizing such catalysts by complexing them with bidentate heterocycles such as 2,2′-bipryridine and 1,10-phenanthroline. Exemplified 1,10-phenanthroline complexes are complexes 5 to 8

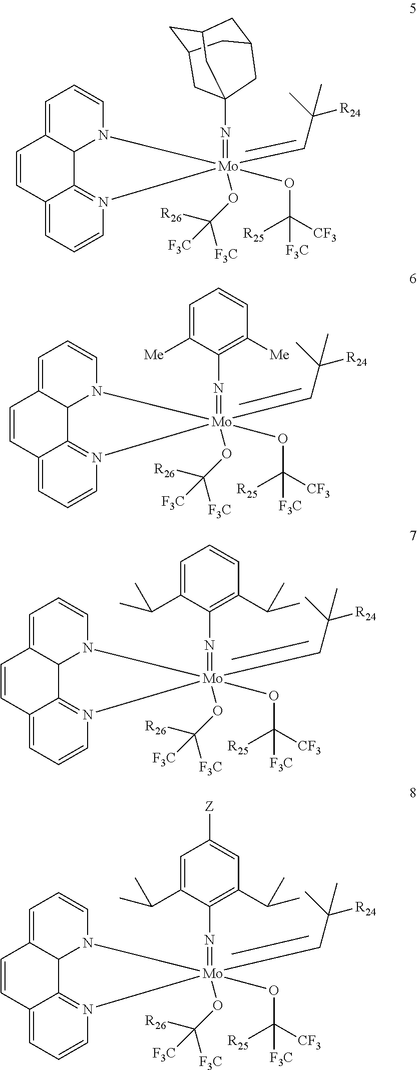

wherein R²⁴=methyl, phenyl; R²⁵, R²⁶=H, methyl, CF₃; Z=methyl, iso-propyl, halogen.

However, such an air stable product is not active catalytically, but the active form of the catalyst must be released by exposure to a Lewis acid such as MgCl₂, MgBr₂, MgI₂, MnCl₂, MnBr₂, MnI₂, FeCl₃, AlCl₃, CuCl₂, ZnCl₂, ZnBr₂, ZnI₂, Zn(triflate)₂ or Zn(trifluoroacetate)₂ and optionally by exposure to heat. Presence of Lewis acids and the formation of adducts of phenanthroline with the Lewis acid may negatively affect the metathesis reaction and generally results in a complex work-up of the reaction mixture in order to isolate the products of the reaction.

EP 3 268 377 B1 discloses tungsten imido alkylidene catalysts stabilized with 1,1 0-phenanthroline. The catalysts must be activated by addition of a Lewis acid such as zinc chloride. Exemplified complexes are complexes III to VI:

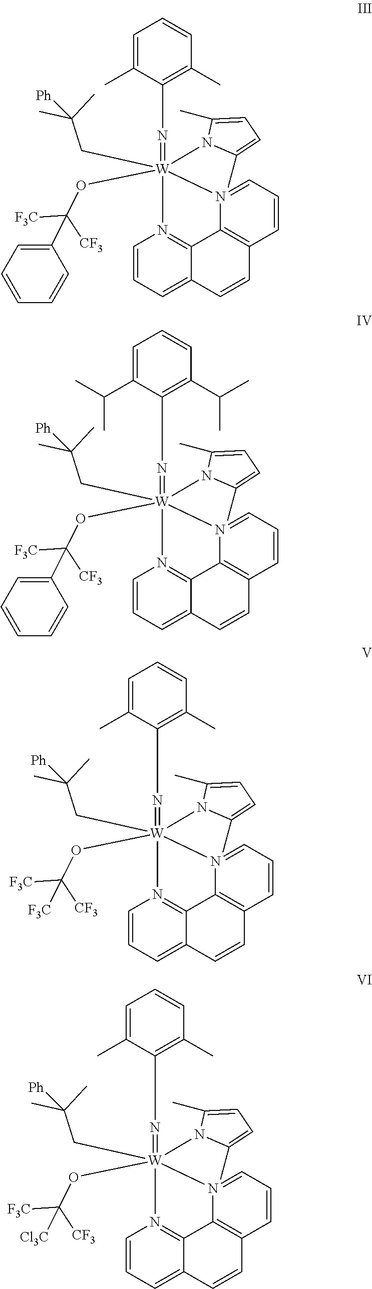

GB 2 537 416 discloses a metathesis catalyst complexed with 2,2′-bipyridine which may be activated by dissolution in a solvent without the need for the addition of a Lewis acid. The inventors of GB 2 537 416 show that these bipyridine adducts are labile and term the spontaneous liberation of the active catalyst as an “autoactivating catalyst”. The inventors conclude that this is the property of a relatively small selection of compounds.

The concept of labile bipyridine adducts has also been published in the scientific literature (Gulyás, H. et al. “Air-stable 18-electron adducts of Schrock catalysts with tuned stability constants for spontaneous release of the active species”, Commun. Chem. 4, 71 (2021); https:/doi.org/10.1038/s42004-021-00503-4).

### OBJECTS OF THE INVENTION

There is an ongoing need in the industry for providing molybdenum and tungsten alkylidene complexes suitable to catalyze olefin metathesis reactions, wherein the complexes have improved air stability, and wherein the complexes do not require chemical activation.

### SUMMARY OF THE INVENTION

This object has been achieved with the 18-electron complexes of formula I defined in independent claim 1. Further independent claims specify a method of making the complexes of formula I, a system comprising the complexes of formula I in equilibrium with their 14-electron complexes of formula II, a method of making the 14-electron complex of formula II from the 18-electron complex of formula I and a method of performing an olefin metathesis reaction using the complexes of formula I. Preferred embodiments are specified in the respective dependent claims.

Without being bound by theory, the inventors discovered that by means of the substitution pattern of the complex of formula I in particular in terms of at least D (1,10-phenanthroline and substituted 1,10 phenanthroline) or C (alkoxide, aryloxide) or D and C the stability constant K of the complex of formula I with respect to the neutral bidentate ligand in a solvent may be adjusted to the range of from 5 L*mol⁻¹ to 250,000 L*mol⁻¹ when measured at 298 K when the complex of formula I is dissolved in the solvent. Thus, this finite stability constant K serves in a selected solvent for an equilibrium between the 18-electron complex comprising the bidentate ligand and which is not active in an olefinic metathesis reaction, and the 14-electron complex from which the bidentate ligand has been released by dissociation, wherein the 14-electron complex is catalytically active in an olefinic metathesis reaction.

Contrary to this, complexes with a virtually infinite stability constant K, i.e., with a stability constant being too high in a predetermined solvent are too stable in order to release the active form therefrom. This applies to the complexes needing the assistance of a Lewis acid in order to remove the bidentate ligand as are known from the prior art as referred to in the Background section.

Complexes having a stability constant being too low, i.e., tending to zero in a predetermined solvent, do not form an 18-electron complex with the neutral bidentate ligand.

The inventors of the present invention discovered in a research program that Schrock-alkylidene complexes of formula I having a stability constant in the range of from 5 L*mol⁻¹ to 250,000 L*mol⁻¹ when measured at 298 K and when the complex of formula I is dissolved in the solvent, are air-stable and catalytically active in an olefinic metathesis reaction without the necessity of removing the bidentate ligand by means of a Lewis acid and hence unnecessarily forming the corresponding by-products, i.e., such complexes are autoactivating. This is a remarkable improvement in view of the cited prior art as discussed in the Background section.

It is noteworthy to mention that the finding of the inventors of GB 2 537 416 A that Schrock-alkylidene complexes bearing a 2,2′-bipyridine as neutral ligand which may be activated by dissolution in a solvent without the need for the addition of a Lewis acid and that this is the property of a relatively small selection of compounds is explainable. Without being bound by theory, the present inventors assume that the diminished complexation capacity of 2,2′-bipyridine compared to that of the 1,10-phenanthroline ligand originates from the conformational flexibility of the former, and from the fact that in the thermodynamically most stable conformer of the bipyridine the torsion angle of the pyridine rings is ca. 40°, hence is suboptimal for bidentate complex formation. This has been confirmed by the above-referenced Gulyás reference.

Contrary to this, the rigid scaffold of 1,10-phenanthroline is optimal for bidentate-complex formation.

The invention relates to the following items:
1. Complex of formula I

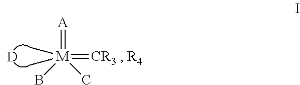

wherein
M=Mo or W;
A is selected from N—R¹ or O, wherein R¹ is C₁₋₁₀ alkyl or aryl, optionally respectively substituted;
B is selected from pyrrole and pyrazole, optionally respectively substituted; or B is C;
C is selected from O—R², wherein R² is C₁₋₁₀ alkyl or aryl, optionally respectively substituted;
D is a neutral bidentate ligand, wherein said ligand is 1,10-phenanthroline or substituted 1,10-phenanthroline;
R³ and R⁴ are independently H, C₁₋₁₀ alkyl or aryl, C₁₋₁₀ alkyl and aryl being optionally substituted; and wherein only one of R³ and R⁴ is hydrogen.
2. The complex of item 1, wherein the complex of formula I is characterized by a stability constant K with respect to the neutral ligand in a solvent, and wherein the substitution pattern of the complex of formula I in terms of at least one of D or C or D and C is selected such to adjust the stability constant K to a range of from 5 L*mol⁻¹ to 250,000 L*mol⁻¹ when measured at 298 K when the complex of formula I is dissolved in the solvent; preferably wherein the substation pattern of at least one of D or D and C is selected such to adjust the stability constant K to a range of from 5 L*mol⁻¹ to 250,000 L*mol⁻¹ when measured at 298 K when the complex of formula I is dissolved in the solvent.
3. Complex of item 1 or ₂, wherein
   R¹ is C₁₋₁₀ alkyl or phenyl, respectively independently substituted with one or more of C₁₋₁₀ alkyl, C₁₋₁₀ alkoxy, phenyl, halogen, CN, and CF₃.
4. Complex of any one of items 1 to ₃, wherein B is pyrrole and pyrazole, respectively independently substituted with one or more of C₁₋₁₀ alkyl, C₁₋₁₀ alkoxy or phenyl.
5. Complex of any one of items 1 to 4, wherein R² is selected from C₁₋₁₀ alkyl, independently substituted with one or more of halogen or phenyl; or is
   phenyl, independently substituted with one or more of C₁₋₁₀ alkyl, C₁₋₁₀ alkyl substituted with one or more of halogen, C₁₋₁₀ alkoxy, phenyl, halogen, —(CH₂)₄— to form an annulated ring with said phenyl, or —(CH═CH—CH═CH)— to form an annulated ring with said phenyl, or with —O-silyl.
6. Complex of any one of items 1 to 5, wherein phenanthroline is independently substituted with one or more electron-donating substituents, preferably selected from C₁₋₁₀ alkyl, C₁₋₁₀ alkoxy, phenyl, or
   is substituted with one or more electron-withdrawing substituents, preferably selected from halogen, CN, CF₃ and CCl₃.
7. Complex of any one of items 1 to 6, wherein R³ and R⁴ are independently H, C₁₋₁₀ alkyl or aryl, wherein C₁₋₁₀ alkyl or aryl are independently substituted with one or more of C₁₋₅ alkyl, C₁₋₅ alkyl substituted with one or more of halogen, C₁₋₅ alkoxy, phenyl, halogen.
8. Complex of any one of items 2 to 7, wherein K is in the range of from 10 L*mol⁻¹ to 150,000 L*mol⁻¹ or 10 L*mol⁻¹ to 100,000 L*mol⁻¹ or 10 L*mol⁻¹ to 50,000 L*mol⁻¹ or 10 L*mol⁻¹ to 10,000 L*mol⁻¹ or 10 L*mol⁻¹ to 5,000 L*mol⁻¹ or 10 L*mol⁻¹ to 500 L*mol⁻¹.
9. Complex of any one of items 2 to 8, wherein the concentration of the complex in the solvent is in the range of from 0.0001 to 0.5 M.
10. Complex of any one of items 2 to 9 wherein 1,10-phenanthroline and substituted 1,10-phenanthroline are selected in order to adjust K.
11. Complex of any one of items 2 to 10, wherein 1,10-phenanthroline is substituted with one or more electron-donating groups in order to increase K compared to a complex of formula I having the same substitution pattern in terms of A, B and C but in which the bidentate ligand D is unsubstituted 1,10-phenanthroline.
12. Complex of any one of items 2 to 10, wherein 1,10-phenanthroline is substituted with one or more electron-withdrawing groups in order to decrease K compared to a complex of formula I having the same substitution pattern in terms of A, B and C but in which the bidentate ligand D is unsubstituted 1,10-phenanthroline.
13. Complex of any one of items 2 to 12, wherein C is selected in order to adjust K, wherein C is characterized in terms of its alkoxide cone angle, provided C is an alkoxide, the alkoxide cone angle and the determination thereof being defined in the description.
14. Complex of item 13, wherein the alkoxide cone angle is increased in order to decrease K compared to a complex having the same substitution pattern in terms of A, B and D; or wherein the alkoxide cone angle is decreased in order to increase K compared to a complex having the same substitution pattern in terms of A, B and D.
15. Complex of any one of items 2 to 12, wherein C is selected in order to adjust K, wherein C is characterized in terms of its steric bulk, provided C is an aryloxide.
16. Complex of item 15, wherein the steric bulk is increased in order to decrease K compared to a complex having the same substitution pattern in terms of A, B and D; or wherein the steric bulk is decreased in order to increase K compared to a complex having the same substitution pattern in terms of A, B and D.
17. Complex of any one of items 1 to 16 wherein the complex is of formula

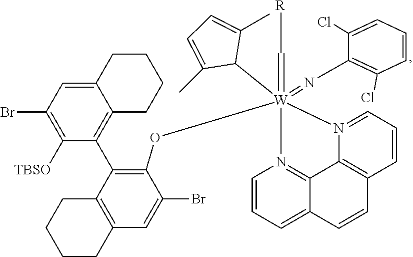

wherein R is C(CH₃)₃, C(CH₃)₂C₆H₅, C₆H₅, or o-(C₁₋₄)-alkoxy C₆H₄;
preferably wherein R is o-(C₁₋₄)-alkoxy C₆H₄;
more preferably wherein the complex is

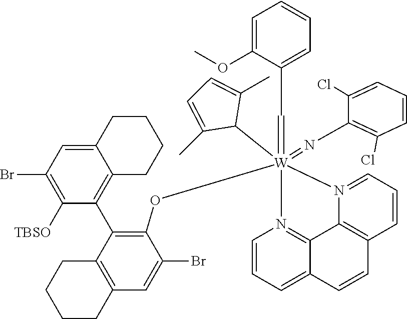

[O-TBS=O-Si(t-butyl)(Me)₂].
18. Method of making a complex as defined in any one of items 1 to 17, comprising:
   subjecting a complex of formula II

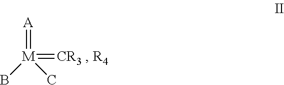

wherein A, B, C, R³ and R⁴ have the meaning as defined with respect to the complex of formula I, to
1,10-phenanthroline or substituted 1,10-phenanthroline,
in the solvent.
19. Method of item 18, further comprising isolating the complex of formula I in solid form.
20. Method of item 18 or 19, wherein the complex of formula I is isolated by filtration.
21. Method of item 18 or 19, wherein the complex of formula I is isolated by evaporating the solvent.
22. System comprising
   a complex of formula I as defined in any one of items 1 to 17 and
   a complex of formula II as defined in item 18;
   wherein the complex of formula I and the complex of formula II are dissolved in the solvent.
23. System of item 22, wherein the system does not comprise a Lewis acid.
24. System of item 23, wherein the Lewis acid is MgCl₂, MgBr₂, Mgl₂, MnCl₂, MnBr₂, MnI₂, FeCl₃, AlCl₃, CuCl₂, ZnCl₂, ZnBr₂, Znl₂, Zn(triflate)₂ or Zn(trifluoroacetate)₂.
25. Method of making a complex of formula II as defined in item 18 comprising:
   dissolving the complex of formula I as defined in any one of items 1 to 17 in the solvent.
26. Method of item 25, wherein the method is performed in the absence of a Lewis acid.
27. Method of item 26, wherein the Lewis acid is MgCl₂, MgBr₂, Mgl₂, MnCl₂, MnBr₂, MnI₂, FeCl₃, AlCl₃, CuCl₂, ZnCl₂, ZnBr₂, Znl₂, Zn(triflate)₂ or Zn(trifluoroacetate)₂.
28. Method of performing a metathesis reaction of a compound comprising an olefinic double bond, comprising:
   adding a complex as defined in any one of items 1 to 17 to the olefinic compound in presence of the solvent.
29. Method of item 28, wherein the compound comprising an olefinic double bond is the solvent.
30. Method of item 28 or 29, wherein the metathesis reaction is performed in absence of a Lewis acid.
31. Method of item 30, wherein the Lewis acid is MgCl₂, MgBr₂, Mgl₂, MnCl₂, MnBr₂, MnI₂, FeCl₃, AlCl₃, CuCl₂, ZnCl₂, ZnBr₂, Znl₂, Zn(triflate)₂ or Zn(trifluoroacetate)₂.

### BRIEF DESCRIPTION OF THE FIGURES

In the figures show

FIG. 1 a schematic drawing of an alkoxide having a O-C_{tertiary} bond and an O-A line segment wherein A is the outermost atom of the group bond to C_{tertiary}. This drawing can be used to calculate the alkoxide cone angle;

FIG. 2 a ¹H NMR spectrum of complex A1 in C₆D₆ (c=0.01 M) at 298 K;

FIG. 3 a ¹H NMR spectrum of complex A2 in C₆D₆ (c=0.01 M) at 298 K;

FIG. 4 a ¹H NMR spectrum of complex A3 in C₆D₆ (c=0.01 M) at 298 K.

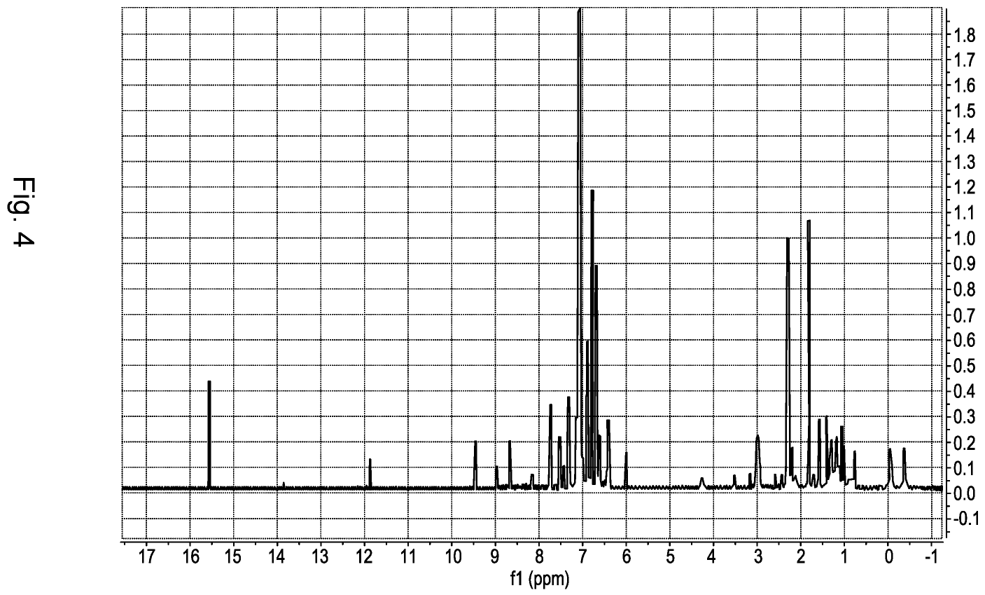

### DETAILED DESCRIPTION OF THE INVENTION

According to a first aspect, the invention relates to a complex of formula I

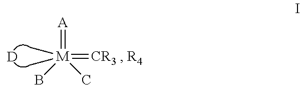

wherein
M=Mo or W;
A is selected from N—R¹ or O, wherein R¹ is C₁₋₁₀ alkyl or aryl, optionally respectively substituted;
B is selected from pyrrole and pyrazole, optionally respectively substituted; or B is C;
C is selected from O—R², wherein R² is alkyl, preferably C₁₋₁₀ alkyl, or aryl, optionally respectively substituted;
R³ and R⁴ are independently H, C₁₋₁₀ alkyl or aryl, C₁₋₁₀ alkyl and aryl being optionally substituted; and wherein only one of R³ and R⁴ is hydrogen;
D is a neutral bidentate ligand, wherein said ligand is 1,10-phenanthroline or substituted 1,10-phenanthroline; preferably wherein the complex of formula I is characterized by a stability constant K with respect to the neutral ligand in a solvent, and wherein the substitution pattern of the complex of formula I in terms of at least one D or C and D is selected such to adjust the stability constant K to a range of from 5 L*mol⁻¹ to 250,000 L*mol⁻¹ when measured at 298 K when the complex of formula I is dissolved in the solvent.

According to the invention, M is Mo or W.

In one embodiment, M is W.

According to the invention, A is selected from N—R¹ or O, wherein R¹ is alkyl, preferably C₁₋₁₀ alkyl, or aryl, optionally respectively substituted.

In a preferred embodiment, A is N—R¹, wherein R¹ is C₁₋₁₀ alkyl or aryl, optionally respectively substituted.

The term “alkyl or C₁₋₁₀ alkyl” as used herein, e.g. used for the definition of R¹, encompasses straight, branched, cyclic and alicyclic alkyl. A preferred C₁₋₁₀ alkyl is C₁₋₅ alkyl. In another embodiment, C₄₋₁₀ alkyl is preferred.

In one embodiment, R¹ is t-butyl or 1-adamantyl.

The term “aryl” as used herein, e.g. used for the definition of R¹, encompasses phenyl, naphthyl, anthracenyl, and phenanthryl, optionally respectively substituted.

Suitable substituents may be selected from one or more of C₁₋₁₀ alkyl, C₁₋₁₀ alkoxy, phenyl, halogen, CN, and CF₃.

Phenyl as aryl is preferred.

In one embodiment, R¹ is C₁₋₁₀ alkyl or phenyl, respectively independently substituted with one or more of C₁₋₁₀ alkyl, C₁₋₁₀ alkoxy, phenyl, halogen, CN, and CF₃.

According to the invention, B is selected from pyrrole and pyrazole, optionally respectively substituted; or

### B is C.

In one embodiment, B is pyrrole and pyrazole, respectively independently substituted with one or more of C₁₋₅ alkyl, C₁₋₅ alkoxy or phenyl.

According to the invention, C is selected from O—R², wherein R² is C₁₋₁₀ alkyl or aryl, optionally respectively substituted.

In one embodiment, R² is selected from C₁₋₅ alkyl, independently substituted with one or more of halogen or phenyl; or is

phenyl, independently substituted with one or more of C₁₋₅ alkyl, C₁₋₅ alkyl substituted with one or more of halogen, C₁₋₅ alkoxy, phenyl, halogen, —(CH₂)₄— to form an annulated ring with phenyl, and —(CH═CH—CH═CH)— to form an annulated ring with phenyl, or with O-silyl.

The term “silyl” may be any silyl group forming a covalent bond between silicon and oxygen.

Suitable silyl groups are e.g. t-butyldimethylsilyl (TBS, TBDMS), trimethylsilyl (TMS), triethylsilyl (TES), triisopropylsilyl (TIPS), t-butyldiphenylsilyl (TBDPS), and triphenylsily.

According to the invention, D is a neutral bidentate ligand, wherein said neutral ligand is 1,10-phenanthroline or substituted 1,10-phenanthroline. Both N atoms of the phenanthroline scaffold bind to M, thus forming the bidentate ligand.

In one embodiment, phenanthroline is substituted with one or more electron-donating groups.

In one embodiment, 1,10-phenanthroline is independently substituted with one or more of C₁₋₅ alkyl, C₁₋₅ alkoxy, —O—(CH₂)ₙ—O—(n=1 or 2), and phenyl. These groups represent electron-donating groups in the meaning of the invention.

In another embodiment, 1,10-phenanthroine is substituted with one or more electron-withdrawing groups.

In one embodiment, 1,10-phenanthroline is independently substituted with halogen, cyano, CF₃or CCl₃. These groups represent electron-withdrawing groups in the meaning of the invention.

Commercially available phenanthrolines are e.g.

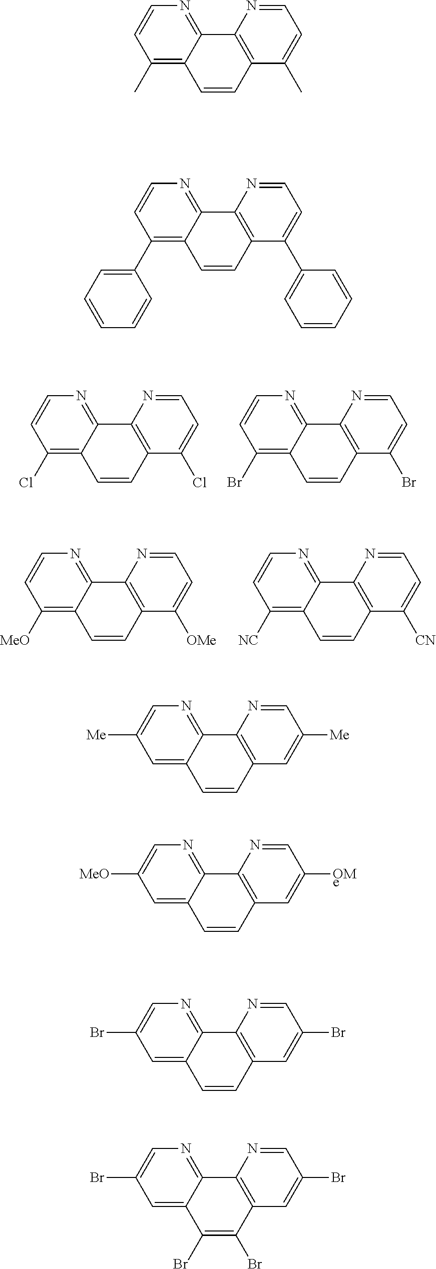

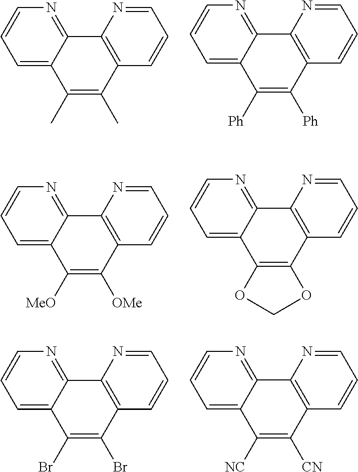

According to the invention, R³ and R⁴ are independently H, C₁₋₁₀ alkyl or aryl, alkyl and aryl being optionally substituted; and wherein only one of R³ and R⁴ is hydrogen.

In one embodiment, R³ and R⁴ are independently H, C₁₋₁₀ alkyl or aryl, wherein C₁₋₁₀ alkyl or aryl are independently substituted with one or more of C₁₋₅ alkyl, C₁₋₅ alkyl substituted with one or more of halogen, C₁₋₅ alkoxy, phenyl, halogen.

According to the invention, the complex of formula I is characterized by a stability constant K with respect to the neutral ligand in a solvent, wherein K is in the range of from 5 L*mol⁻¹ to 250,000 L*mol⁻¹ (5 M⁻¹ to 250,000 M⁻¹) at 298 K.

The term “stability constant” is synonymously used with the term “association constant”. It is the inverse of the dissociation constant. Terms such as “binding constant” and “formation constant” may also synonymously be used for the term “stability constant”.

K can be determined according to known methods employing the law of mass action.

Suitable solvents preferably are organic aromatic solvents such as benzene, toluene, xylene or chlorobenzene, chlorinated hydrocarbons such as dichloromethane or trichloromethane, or the substrate to be metathesized.

According to the invention, the substitution pattern of the complex of formula I in terms of at least D or C and D is selected such to adjust the stability constant K of the complex of formula I with respect to the neutral bidentate ligand in a solvent to a range of from 5 L*mol⁻¹ to 250,000 L*mol⁻¹ when measured at 298 K when the complex of formula I is dissolved in the solvent.

According to the invention, K is in the range of from 5 to 250,000 L*mol⁻¹ when measured at 289 K when the complex of formula I is dissolved in the solvent.

In one embodiment, K is adjusted to a range of from 10 L*mol⁻¹ to 150,000 L*mol⁻¹ or 10 L*mol⁻¹ to 100,000 L*mol⁻¹ or 10 L*mol⁻¹ to 50,000 L*mol⁻¹ or 10 L*mol⁻¹ to 10,000 L*mol⁻¹ or 10 L*mol⁻¹ to 5,000 L*mol⁻¹ or 10 L*mol⁻¹ to 500 L*mol⁻¹.

In one embodiment, the concentration of the complex in the solvent is in the range of from 0.0001 to 0.5 M (0.0001 to 0.5 mol/L).

In one embodiment, 1,10-phenanthroline and substituted 1,10-phenanthroline are selected in order to adjust K.

In one embodiment, 1,10-phenanthroline is substituted with one or more electron-donating groups in order to increase K compared to a complex of formula I having the same substitution pattern in terms of A, B, and C but in which the bidentate ligand D is unsubstituted 1,10-phenanthroline.

In another embodiment, 1,10-phenanthroline is substituted with one or more electron-withdrawing groups in order to decrease K compared to a complex of formula I having the same substitution pattern in terms of A, B and C but in which the bidentate ligand D is unsubstituted 1,10-phenanthroline.

In another embodiment, C is selected in order to adjust K, wherein C is characterized in terms of its alkoxide cone angle, provided C is an alkoxide.

A cone angle has been defined by C. A. Tolman in J. Am. Chem. Soc., 1970, 92, 2956-2965 and Chem. Rev., 77, 313 (1977) with respect to a metal as apex.

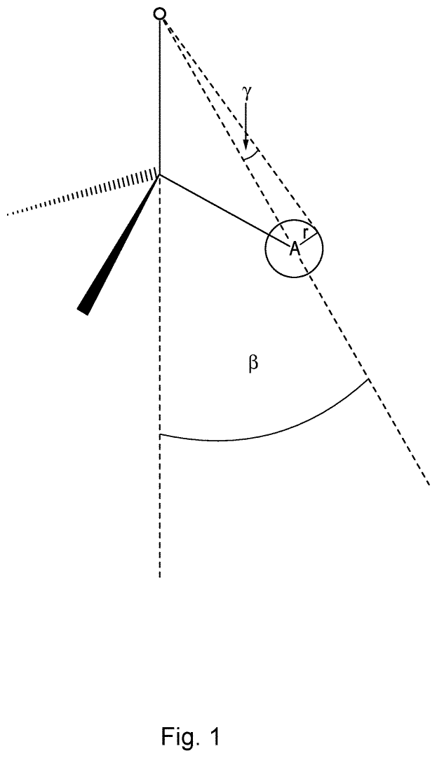

The cone angle as used in this disclosure with respect to C is defined such that the oxygen in C binding the alkyl moiety to M is the apex as shown in FIG. 1. This cone angle a is determined according to the following steps 1 to 4:

1. Find the most stable confirmation of the alkoxide ligand in the desired complex.

2. Determine 1 angles from the structure. β is the angle between the O-C_{tertiary} bond and O-A line segment wherein A is the outermost atom of the group bond to C_{tertiary}. (Determining the R angles consider the center of the O, C_{tertiary} and A atom).

3. Determine y angles from the O-A distance and from r, the Van der Waals radius of A. Sin γ=r/A.

4. If the three organic ligands in the alkoxide are the same, the alkoxide has C₃, symmetry, then a regular cone will cover the ligand. In that case α=2((β+γ).

In case of three different ligands, the steric parameter is defined as the average of the cone angles of the C₃ᵥ symmetric alkoxides of each ligand. Technically, the average of the three β+γ half angles is taken, and the average half angle is multiplied with two as in the following equation:

Cone angle α=2*(β1 +γ1+β2+γ2+β3+γ3): 3

The cone angle is used as a measure for the bulkiness of an alkoxide ligand.

In one embodiment, typically, the increase of the alkoxide cone angle results in the decrease of K compared to a complex having the same substitution pattern in terms of A, B and D.

In another embodiment, typically, the decrease of the alkoxide cone angle results in the increase of K compared to a complex having the same substitution pattern in terms of A, B and D.

Accordingly, in a preferred embodiment, K can be adjusted by C in terms of its cone angle, provided C is an alkoxide.

In a preferred embodiment, the alkoxide cone angle is in the range of from 140° to 225°, preferably 150 to 220°.

In another preferred embodiment, K can be adjusted by C in terms of its steric bulk, provided C is an aryloxide.

The term “steric bulk” means the spatial expansion of C.

In one embodiment, the steric bulk is increased in order to decrease K compared to a complex having the same substitution pattern in terms of A, B and D.

In another embodiment, the steric bulk is decreased in order to increase K compared to a complex having the same substitution pattern in terms of A, B and D.

In a preferred embodiment, both 1,10-phenanthroline, substituted 1,10-phenanthroline in terms of electron-donating groups and electron-withdrawing groups, and C characterized in terms of its alkoxide cone angle or the aryloxide steric bulk are used in order to adjust K.

According to the invention, when K is in the range as defined, there is no need to add a Lewis acid such as zinc chloride to the complex of formula I in order to remove the bidentate ligand from the complex to form the activated 14-electron complex from the 18-electron complex. Rather, the complex of formula I is autoactivating, i.e., without the need of chemical activation.

In a preferred embodiment, the complex is of formula

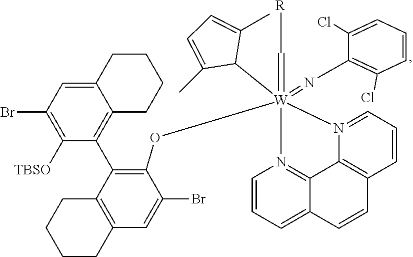

wherein R is C(CH₃)₃, C(CH₃)₂C₆H_{5,} C₆H₅, or o-(C₁₋₄)-alkoxy C₆H₄;
preferably wherein R is o-(C₁₋₄)-alkoxy C₆H₄;
more preferably wherein the complex is

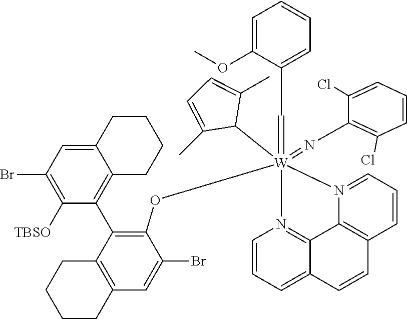

[O-TBS=O-t-butyl-Si(Me)₂].

Since complexes 5 to 8 as defined in WO 2012/116695 and complexes III to VI as defined in EP 3 268 377 B1 are not autoactivating but need chemical activation by a Lewis acid, same may be excluded from item 1 as disclosed in the Summary section.

In a second aspect, the invention relates to a method of making a complex of formula I as defined in any one of the embodiments of the first aspect, comprising:
subjecting a complex of formula II

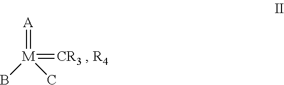

wherein A, B, C, R³ and R⁴ have the meaning as defined with respect to the complex of formula I, to
1,10-phenanthroline or substituted 1,10-phenanthroline.
in the solvent.

In one embodiment, the method further comprises:

isolating the complex of formula I in solid form.

In one embodiment, isolation may be achieved by allowing the complex of formula I to precipitate from the solvent, preferably in the form of crystals. Accordingly, the complex of formula I may be isolated by filtration.

In another embodiment, concentrating the solution inherently results in shifting the equilibrium towards the adduct formation.

Accordingly, in another embodiment, the method further comprises: isolating the complex of formula I by evaporating the solvent.

In a third aspect, the invention relates to a system comprising
a complex of formula I as defined in any one of the embodiments of the first aspect, and
a complex of formula II as defined in the second aspect,
wherein the complex of formula I and the complex of formula II are dissolved in the solvent.

This means nothing else than that complexes of formula I and formula II are in equilibrium with one another. This equilibrium depends on concentration and temperature. Typically, increasing the temperature shifts this equilibrium towards the complex of formula II. Further typically, diluting the solution also shifts this equilibrium towards the complex of formula II.

According to the invention, said system does not contain a Lewis acid.

The term “Lewis acid” as used herein encompasses any Lewis acid which is capable of removing 1,10-phenanthroline from any of complexes defined in WO 2012/116695 and EP 3 268 377 B1.

Lewis acids are e.g. MgCl₂, MgBr₂, MgI₂, MnCl₂, MnBr₂, MnI₂, FeCl₃, AlCl₃, CuCl₂, ZnCl₂, ZnBr₂, ZnI₂, Zn(triflate)₂ or Zn(trifluoroacetate)₂.

In a preferred embodiment, the Lewis acid is zinc chloride.

In a fourth aspect, the invention relates to a method of making a complex of formula II as defined in the second aspect comprising:

dissolving the complex of formula I as defined in any one of the embodiments of the first aspect in the solvent.

In a fifth aspect, the invention relates to a method of performing a metathesis reaction of a compound comprising an olefinic double bond, comprising:

adding a complex as defined in any one of the embodiments of the first aspect to the olefinic compound in presence of the solvent.

In one embodiment, the compound comprising an olefinic double bond is the solvent.

According to the invention, said metathesis reaction is performed in absence of a Lewis acid.

Lewis acids are e.g. MgCl₂, MgBr₂, MgI₂, MnCl₂, MnBr₂, Mnl₂, FeCl₃, AlCl₃, CuCl₂, ZnCl₂, ZnBr₂, ZnI₂, Zn(triflate)₂ or Zn(trifluoroacetate)₂.

In a preferred embodiment, the Lewis acid is zinc chloride.

### EXEMPLIFICATION

Adducts 20-23 of Scheme 1 are representative examples of the complexes according to the invention. They are easy to prepare and to isolate starting from the corresponding bispyrrolide precursor. In fact, the isolation, purification of the 14-electron tungsten-alkylidenes 16-19 is significantly more difficult and lower-yielding, therefore, the adduct formation also has considerable benefits from a synthetic point of view.

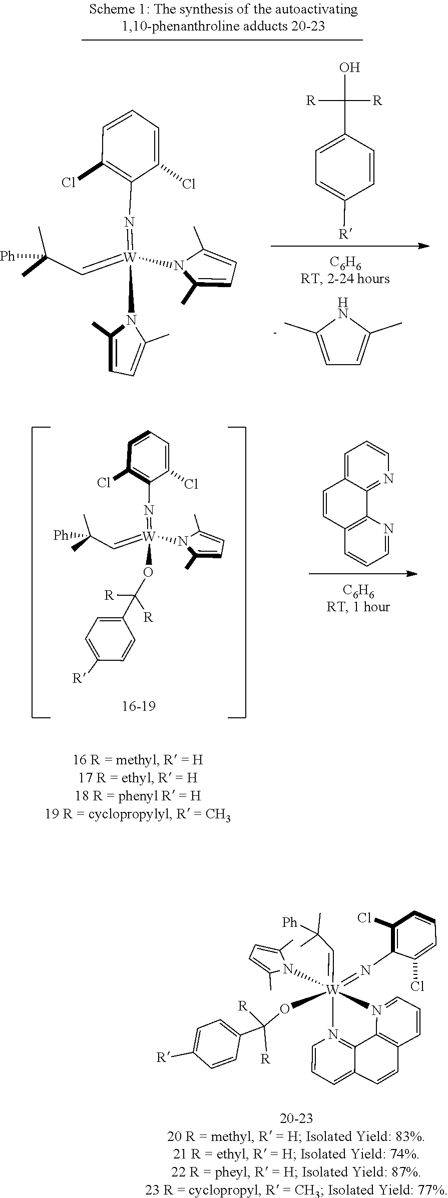

Upon dissolving the 18-electron adducts 20-23, 1,10-phenanthroline is released, i.e. the complex dissociates. Under given conditions, the degree of dissociation will depend on the stability constants of the adducts as shown in Scheme 2 below. The examples demonstrate how the steric bulk of the alkoxide ligand affects the thermodynamic stability of the adducts: with increasing steric bulk of the ligand the stability constant decreases, which will result in a greater degree of dissociation under identical conditions.

The equilibrium can readily be controlled with such extensive physical properties as the concentration and the temperature as demonstrated by NMR spectroscopy.

For instance, the labile (autoactivating) complex 22, having a stability constant K_{22, d6-benzene, 298K}=615 M⁻¹, will liberate 34% of the corresponding active MAP complex 18 in a 0.01 M C₆D₆ solution. Upon diluting the de-benzene solution of 22 to [W]=0.0025 M concentration, 52% of the 14-electron MAP complex will be liberated.

The more labile phenanthroline adduct 23 with a stability constant K_{23, d6-benzene, 298K}=255 M⁻¹ will dissociate to a greater extent under similar conditions. At 25° C., in a 0.01 M C₆D₆ solution of 23, 54% of the active MAP complex 19 is liberated, and 10-fold dilution of the solution practically will fully liberate 19.

It is also important to understand that since the Gibbs free energy of the association is around zero (equilibrium), and the entropy change is negative (association), the enthalpy change of the adduct formation can be expected to be negative. (see the above-referenced Gulyás reference). This means that the dissociation of the phenanthroline from the 18-electron complex, the liberation of the 14-electron MAP complexes can be facilitated by increasing the temperature.

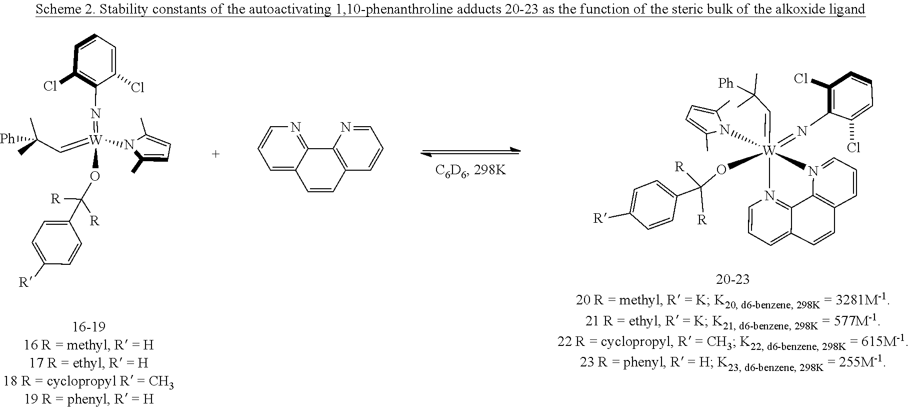

Aryloxide ligands have been proven to be highly important structural motifs in olefin metathesis catalysts. In the design of Schrock catalysts, in general, they seem to be superior to alkoxides in terms of both activity and (stereo)selectivity. The “phenanthroline approach” as disclosed in this invention towards air-stable autoactivating storage complexes can be applied to this class of Schrock catalysts, as well.

MAP complex 24 has been proven very active, highly cis-selective and fairly robust in a number of cross-metathesis reactions of great importance. MAP 24 readily reacts with 1,10-phenanthroline. The corresponding adduct 25 can be isolated in very high, from 70% to >98%, yields depending on the conditions of the reaction and the isolation as shown in Scheme 3.

Importantly, unlike in the previous examples, in the case of 24, the coordination is not completely stereoselective. Based on the alkylidene range of the NMR spectrum of 25, at least two of the possible stereoisomers are being formed and are present in the solution-phase equilibrium. Both the chiral aryloxy ligand and possibly syn-anti isomerism of the alkylidene ligand can give rise to the formation of the observed stereoisomers.

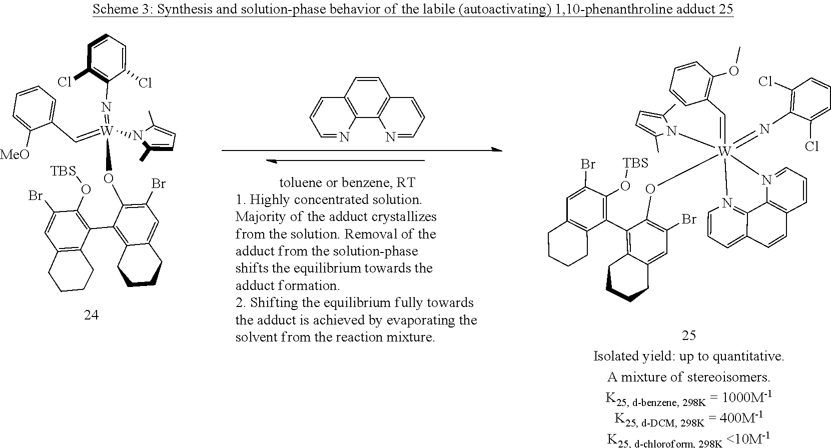

Complex 25 is autoactivating, and its stability constant heavily depends on the nature of the solvent. Under similar conditions, it dissociates considerably more readily in CDCl₂ than in C₆D₆ In CDCl₃ the dissociation is even more favored, below 0.01M concentration, at room temperature, the active MAP complex practically is completely liberated as shown by the stability constants in Scheme 3. As CDCl₃ efficiently reverses the coordination of the phenanthroline, it also serves a proof that all the new alkylidenes formed upon the interaction between 24 and the bidentate N-heterocycle result in stereoisomers of the desired labile coordination compound.

Although the MAP complex 24 is one of the more robust Schrock catalysts, its complexation with 1,10-phenanthroline results in an adduct with markedly increased air-stability. While 0.01 mmol samples of 24 quantitatively decompose in air within 2 hours, in the case of 25, under similar conditions, only 5-10% decomposition was observed in 5 days.

Finally, complex 25, the air-stable 18-electron storage complex of complex 24, has also been proven an extremely efficient catalyst, which does not require the use of Lewis acidic activating agents. It particularly performs well in refining plant oils by ethenolysis, surpassing even, under appropriate conditions, the performance of 24. The catalytic results also prove that the phenanthroline is not only a protecting agent, but it can also be an additional tool to tune and better catalytic performance.

The scheme and table below show the influence of the bulkiness of C expressed in terms of the cone angle on K:

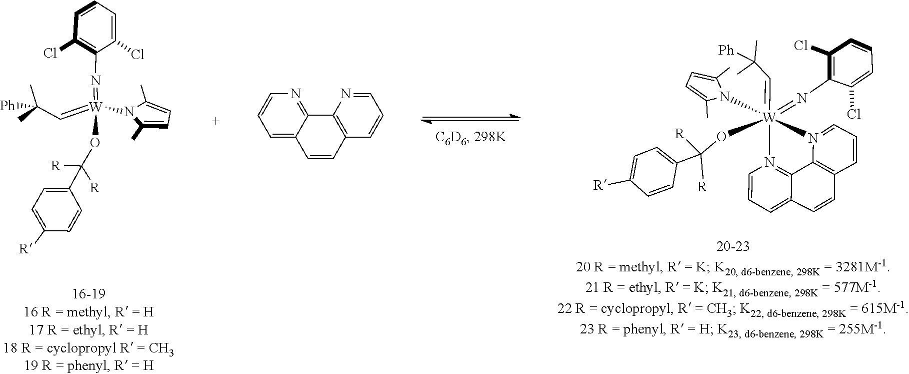

| MAP•PHEN | | Alkoxide | |
|---|---|---|---|
| adduct | ALKOXIDE | Cone Angle | K_{d6-benzene, 298K} |
| 20 | PhMe₂CO⁻ | 182° | 3281 |
| 21 | PhEt₂CO⁻ | 202° | 577 |
| 22 | (4-MeC₆H₄)(cPr)₂CO⁻ | 215° | 615 |
| 23 | Ph₃CO⁻ | 223° | 255 |
| 26PHEN | Ph(iPr)₂CO⁻ | 233° | 0 |

^{a}Alkoxide Cone Angles were determined from the computed and optimized structures of the corresponding MAP complexes 20-23 and 26PHEN

K decreases with increasing cone angle and vice versa. This shows that bulkiness of C can be used to adjust K.

Autoactivating complex 25 was used in homo-cross-metathesis of 9-DAME according to the following equation:

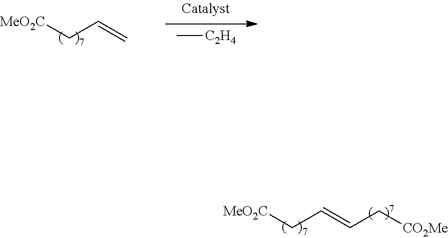

The following table shows results:

| | Loading | | | | |
|---|---|---|---|---|---|
| Purification | (ppm per | | | | |
| method | mole | T | t | Conversion | |
| of 9-DAME | substrate) | [° C.] | [h] | [%] | E/Z |
| Adsorptive | 50 | 50 | 24 | 77 | 9/91 |
| triethyl aluminum | 25 | rt | 4 | 26 | 2/98 |
| treatment | | | | | |

In summary, the inventors of the present invention have discovered in a research program that 1,10-phenanthroline can efficiently be used to synthesize novel air-stable autoactivating storage complexes for 14-electron Schrock catalysts. The complexation not only increases air-stability, but it can also improve the over-all yields of the catalyst synthesis. The 18-electron phenanthroline adducts can directly be used as pre-catalysts in olefin metathesis reactions, without the necessity to remove the phenanthroline from the coordination sphere with Lewis acids such as zinc chloride. On the contrary, the phenanthroline may actually serve as an additional tool to influence the catalytic performance of the alkylidene complexes.

### PREPARATION EXAMPLES

### Example 1: Synthesis of 20

Bispyrrolide precursor W(CHCMe₂Ph)(NAr^{diCl})(Me₂Pyr)₂ (Ar^{diCl}=2,6-dichlorophenyl, Me₂Pyr=2,5-dimethylpyrrolide) (137.4 mg, 0.207 mmol) was dissolved in toluene (10 mL), and the solution was cooled to −30 Celsius in a double-jacketed reactor. PhMe₂COH (28 mg, 0.207 mmol) was added as a solid. The alcohol residues from the walls of the vial were washed into the reaction mixture with toluene (4×0.5mL). The reaction mixture was stirred overnight at −30° C. ¹H NMR analysis of the reaction mixture indicated complete conversion into 16. The reaction mixture was allowed to warm to 0° C., and 1,10-phenantroline (37.4 mg, 0.207 mmol) was added. The reaction mixture turned deep red. It was stirred for six hours without cooling, while the temperature reached RT. ¹H NMR analysis of the reaction mixture showed an equilibirium between the MAP complex and its phenanthroline adduct. The solvent was removed, and the solid residue was stirred with pentane. The suspension was moved into the freezer for a couple of hours, and then the product was isolated by filtration, carried out in the freezer on a precooled frit. Yellowish brown solid. Yield: 152 mg (83%).

NMR characterization of 16: ¹H-NMR (C₆D₆): δ 1.50 (s, 3H, CH₃), 1.56 (s, 3H, CH₃), 1.57 (s, 3H, CH₃), 1.63 (s, 3H, CH₃), 2.34 (s, 6H, CH₃ Me₂Pyr), 6.16 (s br, 2H, CH Me₂Pyr), 6.26 (t, 1H, N—Ar Cₚₐᵣₐ—H), 8.61 ppm (s, 1H, W═CH, ²J_{WH}=16.0 Hz).

NMR characterization of 20: ¹H-NMR (C₆D₆): δ 0.63 (s, 3H, CH₃), 0.98 (s, 3H, CH₃), 1.94 (s, 3H, CH₃ neophylidene), 2.18 (s, 3H, CH₃ neophylidene), 2.88 (br, 6H, CH₃ Me₂Pyr), 6.04 (t, ³J_{HH}=8.0 Hz, 1H, N—Ar Cₚₐᵣₐ—H), 6.55 (dd, J_{HH}=8.1, 4.9 Hz, 1 H, C3-H PHEN), 6.68 (dd, J_{HH}=8.1, 5.1 Hz, 1H, C3′-H PHEN), 6.74-7.41 (m, 16H, Cₘₑₜₐ—H neophylidene, Cₚₐᵣₐ—H neophylidene, CH Me₂Pyr, N—Ar Cₘₑₜₐ—H, C4-H, C4′-H, C5-H, C5′-H PHEN, alkoxy Ph), 7.73 (m, 2H, Cₒᵣₜₕₒ—H), 8.99 (dd, J_{HH}=5.1, 1.4 Hz, 1H, C2′-H PHEN), 9.36 (dd, J_{HH}=4.9, 1.4 Hz, 1H, C2-H PHEN), 12.02 ppm (s, ²J_{WH}=10.0 Hz, 1H, W═CH).

### Example 2: Synthesis of 21

To bispyrrolide precursor W(CHCMe₂Ph)(NAr^{diCl})(Me₂Pyr)₂ (Ar^{diCl}=2,6-dichlorophenyl, Me₂Pyr=2,5-dimethylpyrrolide) (166 mg, 0.25 mmol) dissolved in benzene (3 mL) the corresponding alcohol (0.25 mmol) dissolved in benzene (1 mL) was added gradually, while the reaction mixture was being stirred at room temperature. The progress of the reaction was controlled by ¹H NMR analysis of the reaction mixture. At >98% NMR yield of 17, 1,10-phenanthroline (0.25 mmol) dissolved in benzene (ca. 2 mL) was added in situ, and the reaction mixture was stirred for an hour. The solvent was removed in vacuum, the residue was dissolved in toluene, and the product was isolated by crystallization in the freezer (−38° C.) from a mixture of toluene and pentane. Brownish yellow solid. Yield: 160 mg (74%).

NMR characterization of 17: ¹H-NMR (300 MHz, C₆D₆): δ 0.65 (t, ³J_{HH}=7.4 Hz, 3H, CH₂CH₃), 0.70 (t, ³J_{HH}=7.4 Hz, 3H, CH₂CH₃), 1.66 (s, 3H, CH₃neophylidene), 1.72 (s, 3H, CH₃ neophylidene), 1.74-1.90 (m, 4H, CH₂CH₃), 2.39 (s, 6H, CH₃ Me₂Pyr), 6.15 (s br, 2H, CH Me₂Pyr), 6.28 (t, ³J_{HH}=8.1 Hz, 1H, N—Ar Cₚₐᵣₐ—H), 6.91 (d, ³J_{HH}=8.1 Hz, N—Ar Cₘₑₜₐ—H), 6.96 (m, 1H, Cₚₐᵣₐ—H neophylidene), 7.03 (m, 1H, —OC(C₂H₅)—Ph Cₚₐᵣₐ—H), 7.08-7.23 (m, 6H, Cₘₑₜₐ—H neophylidene, OC(C₂H₅)—Ph Cₒᵣₜₕₒ—H, OC(C₂H₅)—Ph Cₘₑₜₐ—H), 7.44 (m, 2H, Cₒᵣₜₕₒ—H neophylidene), 9.12 ppm (s, 1H, W═CH, ²J_{WH}=16.0 Hz).

NMR characterization of 21: ¹H-NMR (C₆D₆): δ 0.22 (t, ³J_{HH}=7.4 Hz, 3H, CH₂CH₃), 0.41 (t, ³J_{HH}=7.4 Hz, 3H, CH₂CH₃), 0.69 (m, 1H, CH₂CH₃), 1.09 (m, 1H, CH₂CH₃), 1.49 (m, 2H, CH₂CH₃), 2.00 (s, 3H, CH₃ neophylidene), 2.22 (s, 3H, CH₃ neophylidene), 2.74 (w br, 3H, CH₃ Me₂Pyr), 3.21 (w br, 3H, CH₃ Me₂Pyr), 6.04 (t, ³J_{HH}=8.0 Hz, 1H, N—Ar Cₚₐᵣₐ—H), 6.43-6.68 (m, 7H, C3-H, C3′-H, C4′-H, C5-H, C5′-H PHEN, Cₚₐᵣₐ—H neophylidene, —OC(C₂H₅)—Ph Cₚₐᵣₐ—H), 6.79 (s br, 2H, CH Me₂Pyr), 6.86-6.94 (m, N—Ar Cₘₑₜₐ—H), 7.08-7.21 (m, 4H, Cₘₑₜₐ—H, Cₒᵣₜₕₒ—H), 7.25 (dd, J_{HH}=7.9, 1.2 Hz, 1H, C4-H PHEN), 7.32 (m, 2H, Cₘₑₜₐ—H), 7.73 (m, 2H, Cₒᵣₜₕₒ—H), 8.87 (dd, J_{HH}=5.0, 1.2 Hz, 1H, C2-H PHEN), 9.17 (dd, J_{HH}=4.8, 1.3 Hz, 1H, C2-H PHEN), 12.09 ppm (s, ²J_{WH}=10.0Hz, 1H, W═CH).

### Example 3: Synthesis of 22

To bispyrrolide precursor W(CHCMe₂Ph)(NAr^{diCl})(Me₂Pyr)₂ (Ar^{diCl}=2,6-dichlorophenyl, Me₂Pyr=2,5-dimethylpyrrolide) (296 mg, 0.45 mmol) dissolved in benzene (6 mL) dicyclopropyl(para-tolyl)methanol (0.45 mmol) dissolved in benzene (2 mL) was added. The reaction mixture was stirred for 4 hours. ¹H NMR analysis of the reaction mixture showed complete conversion into 18. 1,10-Phenanthroline (0.45 mmol) dissolved in benzene (ca. 4 mL) was added in situ, and the reaction mixture was stirred for an hour. The solvent was removed in vacuum, the residue was triturated in pentane resulting in a brownish yellow solid, which was filtered, washed with pentane, and dried in vacuum. Yield: 305 mg (77%).

NMR characterization of 18: ¹H-NMR (C₆D₆) δ (ppm): 0.1-0.7 (m, 8H, CH₂-cyclopropyl), 0.95-1.1 (m, 2H, CH-cyclopropyl) 1.58 (s, 3H, CH₃), 1.66 (s, 3H, CH₃), 2.13 (s, 3H, Ar—CH₃), 2.33 (s, 12H, CH₃), 6.17 (s, 2H, ═CH Me₂Pyr), 6.27 (t, 1H, N—Ar Cₚₐᵣₐ—H), 6.85-7.13 (m, 10H, aromatic), 7.33 (m, 2H, Cₒᵣₜₕₒ—H neophylidene), 7.44 (m, 2H, Cₒᵣₜₕₒ—H benzyl), 8.67 (s, ²J_{WH}=15.3 Hz, 1H, W═CH).

NMR characterization of 22: ¹H-NMR (C₆D₆): δ-1.70 (m, 1H, CH₂-cyclopropyl), −0.86 (m, 2H, CH₂-cyclopropyl), −0.65 (m, 1H, CH₂-cyclopropyl), −0.52 (m, 1H, CH₂-cyclopropyl), −0.40 (m, 1H, CH₂-cyclopropyl), −0.23 (m, 1H, CH₂-cyclopropyl), 0.16 (m, 1H, CH₂-cyclopropyl), 1.24 (m, 2H, CH-cyclopropyl), 1.96 (s, 3H, CH₃ neophylidene), 2.16 (s, 3H, CH₃), 2.18 (s, 3H, CH₃), 2.81 (w br, 6H, CH₃ Me₂Pyr), 6.06 (t, ³J_{HH}=8.0 Hz, 1H, N—Ar Cₚₐᵣₐ—H), 6.57 (dd, J_{HH}=8.2, 5.0 Hz, 1 H, C3-H PHEN), 6.78 (dd, J_{HH}=8.2, 5.2 Hz, 1H, C3′-H PHEN), 6.87-7.47 (m, 13H, C4-H, C4′-H, C5-H, C5′-H PHEN, Cₘₑₜₐ—H neophylidene, Cₚₐᵣₐ—H neophylidene, CH Me₂Pyr, N—Ar Cₘₑₜₐ—H, Cₘₑₜₐ—H), 7.52 (m, 2H, Cₒᵣₜₕₒ—H), 7.67 (m, 2H, Cₒᵣₜₕₒ—H), 9.20 (dd, J_{HH}=5.2, 1.5 Hz, 1H, C2′-H PHEN), 9.58 (dd, J_{HH}=5.0, 1.5 Hz, 1H, C2-H PHEN), 12.07 ppm (s, ²J_{WH}=10.0 Hz, 1H, W═CH).

A 0.01M solution of the isolated autoactivating complex 22 contained at 298 K in C₆D₆ 33% of liberated complex 18. A 0.0025M solution of the isolated autoactivating complex 22 contained at 298 K in C₆D₆ 52% of liberated complex 18.

### Example 4: Synthesis of 23

To bispyrrolide precursor W(CHCMe₂Ph)(NAr^{diCl})(Me₂Pyr)₂ (Ar^{diCl}=2,6-dichlorophenyl, Me₂Pyr=2,5-dimethylpyrrolide) (141 mg, 0.2125 mmol) dissolved in benzene (3 mL) Ph₃COH (55 mg, 0.2125 mmol, 0.85 equiv.) was added as a solid, and the residue from the walls of the vial was washed into the reaction mixture with benzene (2 mL). The reaction mixture was stirred at room temperature for an hour. ¹H

NMR analysis of the reaction mixture indicated complete conversion into 19. No excess of the alcohol could be detected. 1,10-phenanthroline (38 mg, 0.2125 mmol) was added, and the reaction mixture was stirred for a half an hour at room temperature. ¹H NMR analysis of the reaction mixture showed and equilibirium between the MAP complex and its phenanthroline adduct. All volatiles were removed in vacuum. The residue was triturated with pentane yielding the product as a brownish yellow powder. It was isolated by filtration, washed with small amounts of cold pentane, and dried in vacuum. Yield: 182 mg (87%).

NMR characterization of 19: ¹H-NMR (300 MHz, C₆D₆): δ 1.58 (s, 3H, CH₃ neophylidene), 1.60 (s, 3H, CH₃ neophylidene), 2.24 (s, 6H, CH₃ Me₂Pyr), 6.04 (s br, 2H, CH Me₂Pyr), 6.28 (t, ³J_{HH}=8.1 Hz, 1H, Cₚₐᵣₐ—H N—Ar), 6.91 (d, ³J_{HH}=8.1 Hz, Cₘₑₜₐ—H N—Ar), 6.93 (m, 1H, Cₚₐᵣₐ—H neophylidene), 6.97-7.10 (m, 11H, Cₘₑₜₐ—H OC(Ph)₃, Cₚₐᵣₐ—H OC(Ph)₃, Cₘₑₜₐ—H neophylidene), 7.25-7.34 (m, 8H, Cₒᵣₜₕₒ—H OC(Ph)₃, Cₒᵣₜₕₒ—H neophylidene), 7.92 ppm (s, 1H, W═CH, ²J_{WH}=16.5 Hz).

NMR characterization of 23: ¹H-NMR (C₆D₆): δ 1.96 (5, 3H, CH₃neophylidene), 2.05 (s, 3H, CH₃ neophylidene), 2.54 (s, 3H, CH₃ Me₂Pyr), 2.87 (s, 3H, CH₃ Me₂Pyr), 5.98 (t, ³J_{HH}=8.1 Hz, 1H, N—Ar Cₚₐᵣₐ—H), 6.50 (dd, J_{HH}=8.2, 4.9 Hz, 1H, C3-H PHEN), 6.57 (dd, J_{HH}=8.0, 5.2 Hz, 1H, C3′-H PHEN), 6.33-7.41 (m, 24H, Cₘₑₜₐ—H neophylidene, Cₚₐᵣₐ—H neophylidene, CH Me₂Pyr, N—Ar Cₘₑₜₐ—H, C4-H, C4′-H, C5-H, C5′-H PHEN, aryloxy Ph), 7.52 (m, 2H, Cₒᵣₜₕₒ—H), 7.76 (m, 2H, Cₒᵣₜₕₒ—H), 8.77 (dd, J_{HH}=5.2, 1.4 Hz, 1H, C2′-H PHEN), 9.49 (dd, J_{HH}=4.9, 1.5 Hz, 1H, C2-H PHEN), 12.29 ppm (s, ²J_{WH}=10.5 Hz, 1H, W═CH).

A 0.01M solution of the isolated autoactivating complex 23 contained at 298 K in C₆D₆ 54% of liberated complex 19. A 0.001M solution of the isolated autoactivating complex 23 contained at 298 K in C₆D₆>95% of liberated complex 19.

### Example 5: Synthesis of 25; Synthetic Procedure A

24 (899 mg, 0.8 mmol) and 1,10-phenanthroline (144 mg, 0.8 mmol) were dissolved in benzene (15 mL). The reaction mixture was stirred at room temperature. The orange product started to crystallize within 2 hours, eventually resulting in a thick suspension. The reaction mixture was stirred overnight at room temperature. The product was isolated by filtration, washed with pentane. It was first dried on the frit in vacuum induced N₂ flow. It was transferred into a tared vial, and then it was further dried in high vacuum at room temperature. Bright orange powder. Yield: 807 mg (77%). One single constitutional isomer, but a mixture of stereoisomers.

### Example 5: Synthesis of 25; Synthetic Procedure B

24 (674 mg, 0.6 mmol) and 1,10-phenanthroline (108 mg, 0.6 mmol) were dissolved in benzene (10 mL). The reaction mixture was stirred at room temperature. The orange product started to crystallize within 2 hours, turning gradually into a dense orange suspension. Evaporation of the benzene in vacuum yielded the product as an orange solid. Yield: quantitative. One single constitutional isomer, but a mixture of stereoisomers. (The NMR characteristics of the products obtained via the procedures A and B are identical.)

Selected NMR data of 25: ¹H-NMR (C₆D₆, δ_{ref 1H solvent}=7.16 ppm): 13.19 ppm (broad s, 1H, W═CH, major stereoisomer); 12.99 (broad s, 1H, W═CH, minor stereosiomer).

Compound 25 contained less than 2% of ArOH (C₆D₆, 0.01M, 298 K).

After being in contact with air for 5 days, compound 25 contained ca. 5% of ArOH (C₆D₆, 0.01M, 298 K).

### Example 6: HCM of 9DAME Using Complex 25

Substrate purification of 9-DAME by adsorptive treatment: the substrate was percolated on basic aluminium oxide (20 weight %) three times.

Under the atmosphere of the glovebox in an ovendried 4 mL vial the substrate was added by an automatic pipette and the catalyst stock solution was added to it. The vial was closed by a perforated cap and the reaction mixture was stirred at r.t. Then a sample was taken and dissolved in EtOAc (Suprasolv for GC) and analysed by GCMS to determine conversion.

GCMS-FID system used for the analysis of starting materials and product mixtures: Shimadzu 2010 Plus with split injection method, Column: Zebron ZB-35HT INFERNO, 30 m×0.25 mm×0.25 μm.

Complexes 24 and 25 were used for catalyzing the reaction. Results are presented in the following table:

| | Loading | | | | |
|---|---|---|---|---|---|
| | (ppm | T | t | Conversion | |
| Complex | mole) | [° C.} | [h] | [%] | E/Z |
| 24 | 50 | rt | 24 | 77 | 21/79 |
| 25 | 50 | 50 | 24 | 77 | 9/91 |

### Example 7: Ethenolysis of FAME

In a nitrogen gas filled glovebox, fatty acid methyl ester were measured into 30 mL glass vials and mixed with the stock solution of triethylaluminum (23% wt in toluene). The optimal triethylaluminum amount was determined previously and was found to be 700 ppm. Mixtures were stirred at r.t. for 1 hour. Catalysts 25 were added as a stock solution (0.01 M in benzene) The vial was placed into a stainless steel autoclave equipped with an alublock and was stirred at 50° C. under 10 atm of ethylene gas overpressure for 18 hours. Five reactions were performed in the same autoclave with common gas space. The excess of ethylene was let out. From the reaction mixture 2.0 μl was taken out and diluted to 1.5 ml with n-pentane and analyzed by GCMS-FID, (Shimadzu 2010 Plus, column: Zebron ZB-35HT INFERNO, 30 m×0.25 mm×0.25 μm.

Results are presented in the following table:

| | | | | Total | | | |
|---|---|---|---|---|---|---|---|
| | Loading | Loading | Molar ratio | Conversion | Yield | 9-DAME | |
| Complex | (ppmwt) | (ppmmole) | Substrate/catalyst | [%] | [%] | selectivity | TON |
| 25 | 50 | 11 | 88898 | 76 | 63 | 0.8 | 56273 |

### Example 8: Synthesis of Complex A1

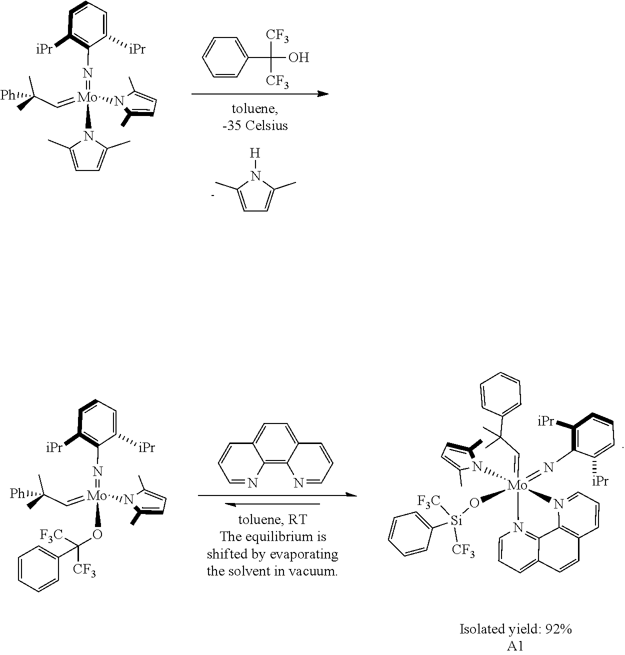

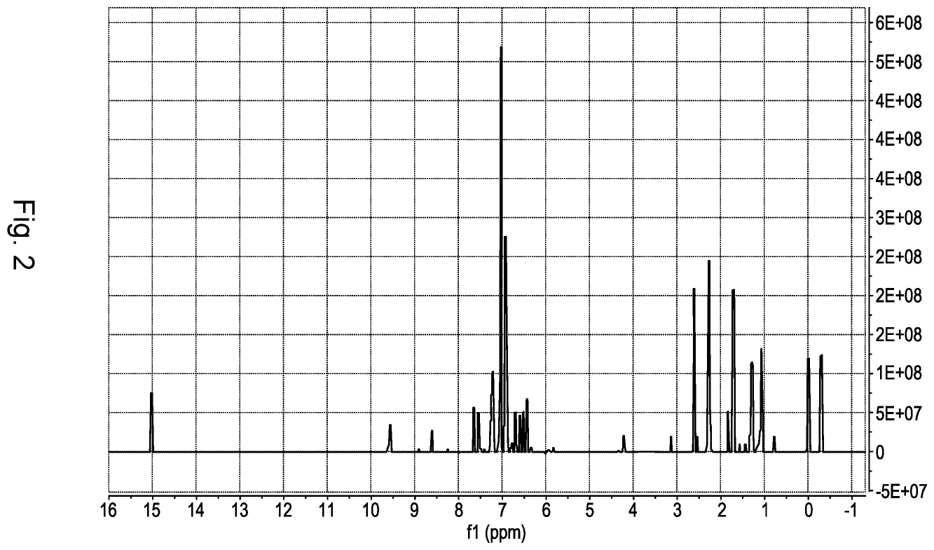

The bispyrrolide precursor Mo(CHCMe₂Ph)(NAr^{diiPr})(Me₂Pyr)₂ (Ar^{diiPr}=2,6-diisopropylphenyl, Me₂Pyr=2,5-dimethylpyrrolide) (118 mg, 0.2 mmol) was dissolved in benzene (2 mL). Ph(CF₃)₂COH (30 microL, 43.6 mg, 0.179 mmol) was added. The reaction mixture was stirred at room temperature overnight. NMR analysis confirmed the formation of the desired MAP complex together with a small amount of the corresponding bisalkoxide complex as a side-product. 1,10-Phenanthroline (32.3, 0.179 mmol) was added to the reaction mixture. The residues of the phenanthroline were rinsed into the reaction mixture from its vial with small amounts of benzene (ca. 1 mL in total). The reaction mixture was stirred at RT for an hour, and then all volatiles were evaporated in vacuo. The residue was triturated in pentane, yielding the product as an orange powder. It was isolated by filtration and dried in vacuo. Orange solid. Yield: 151 mg (92%). ¹H NMR: 15.03 ppm; ¹⁹F NMR: −69.78 (q), −75.87 (q) ppm. Complete ¹H NMR (C₆D₆) see FIG. 2.

### Example 9: Synthesis of Complex A2

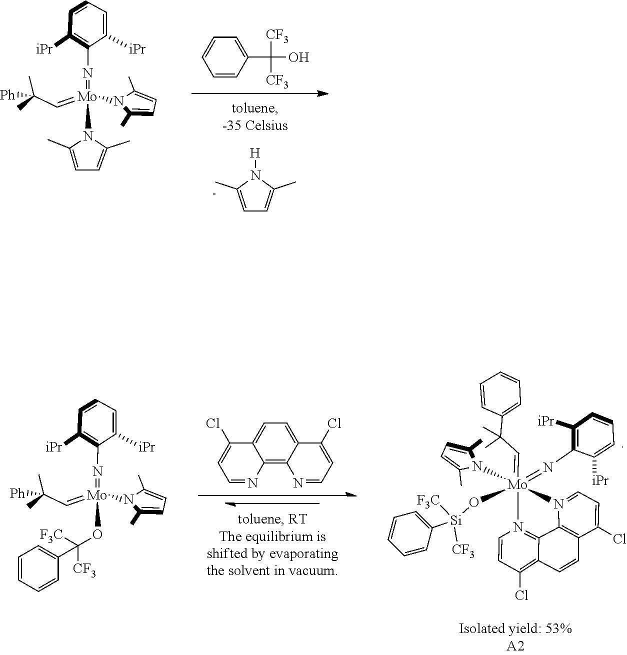

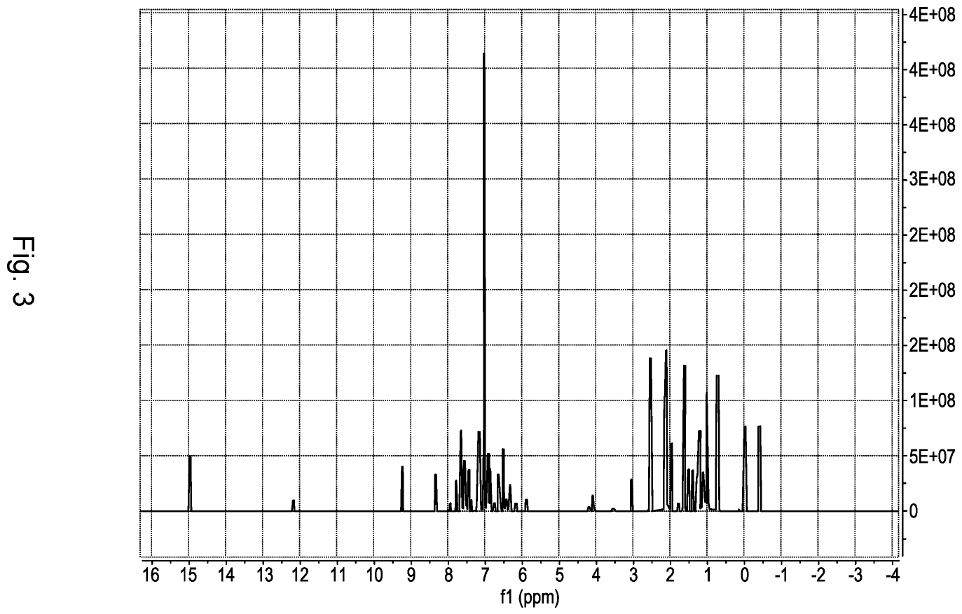

The bispyrrolide precursor Mo(CHCMe₂Ph)(NAr^{diiPr})(Me₂Pyr)₂ (Ar^{diiPr}=2,6-diisopropylphenyl, Me₂Pyr=2,5-dimethylpyrrolide) (118 mg, 0.2 mmol) was dissolved in toluene (3 mL). The solution was transferred into the glovebox freezer and left to cool to −30° C. A toluene solution of Ph(CF₃)₂COH (0.7 mL, 0.12M, 0.084 mmol) precooled in the freezer to −30° C. was added to the solution of the Mo-bispyrrolide precursor. The reaction mixture was hand-stirred for a few seconds to homogenize the reaction mixture, and then it was left in the freezer without stirring. In an hour, another portion of the Ph(CF₃)₂COH solution (0.7 mL, 0.12M, 0.084 mmol) was added, and the reaction mixture was left in the freezer for another hour without stirring. Then the reaction mixture was removed from the freezer, and it was stirred for 3 hours at room temperature. NMR analysis revealed that the reaction mixture contained both unreacted Mo-bispyrrolide and alcohol. It was stirred at room temperature overnight and analyzed again by NMR. The alcohol was consumed, and ca. 8% of the bispyrrolide remained unreacted. The reaction mixture was cooled to −30 Celsius, and a third portion of the Ph(CF₃)₂COH solution (0.110 mL, 0.12M, 0.0132 mmol) was added to the reaction mixture. The reaction mixture was left in the freezer for an hour, and then it was stirred at room temperature for 6 hours. NMR analysis revealed that the Mo-bispyrrolide content decreased to ca. 3%. To the solution of the MAP complex, 4,7-dichloro-1,10-phenanthroline (47.1 mg, 0.189 mmol; carefully dried previously in DCM solution, with molecular sieves) was added. The reaction mixture turned dark reddish-brown. Residues of the phenanthroline were washed into the reaction mixture from its vial with small amounts of toluene (2×0.5 mL). The reaction mixture was stirred until the dichloro-phenanthroline was completely dissolved (ca. 30 min), and then all volatiles were evaporated in vacuo. The triturating the residue in pentane yielded the product as a yellowish-brown solid. It was isolated by filtration and washed with pentane. It was dried, first in vacuum-induced argon flow, and then in vacuo. Yellowish-brown solid. Yield 95 mg (53%). ¹H NMR: 14.96 ppm; ¹⁹F NMR: −69.78 (q), −75.87 (q) ppm. Complete ¹H NMR (C₆D₆) see FIG. 3.

### Example 10: Synthesis of Complex A3

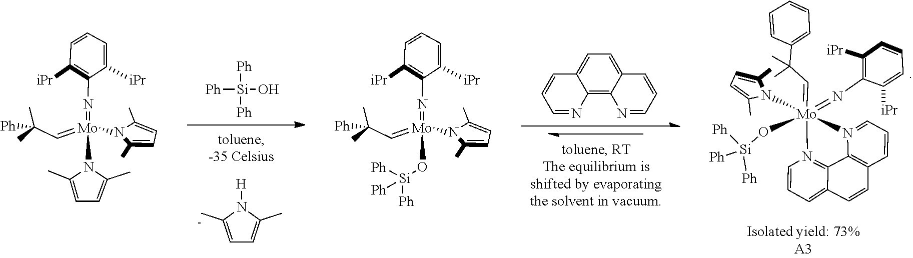

The bispyrrolide precursor Mo(CHCMe₂Ph)(NAr^{diiPr})(Me₂Pyr)₂ (Ar^{diiPr}=2,6-diisopropylphenyl, Me₂Pyr=2,5-dimethylpyrrolide) (59 mg, 0.1 mmol) was dissolved in toluene (2 mL). The solution was transferred into the glovebox freezer and left to cool to −30° C. A toluene solution of Ph₃SiOH (27.6 mg, 0.1 mmol in 2 mL of toluene) precooled in the freezer to −30° C. was added to the solution of the Mo-bispyrrolide precursor. The reaction mixture was hand-stirred for a few seconds to homogenize the reaction mixture, and then it was left in the freezer without stirring for 48 hours. NMR analysis confirmed the complete and selective conversion of the Mo-bispyrrolide precursor into the desired MAP complex. 1,10-Phenanthroline (17.1 mg, 0.095 mmol) dissolved in toluene (1 mL) was added to the reaction mixture. The residues of the phenanthroline were rinsed into the reaction mixture from its vial with small amounts of benzene (ca. 1 mL in total). The reaction mixture was stirred at RT for an hour, and then all volatiles were evaporated in vacuo. The residue was triturated in pentane, yielding the product as a dark yellow powder. It was isolated by filtration and dried in vacuo. Dark yellow solid. Yield: 68 mg (75%). ¹H NMR: 15.54 ppm. Complete ¹H NMR (C₆D₆) see FIG. 3.

1. Complex of formula I 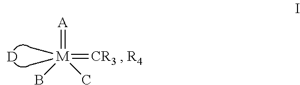
wherein
M=Mo or W;
A is selected from N—R¹ or O, wherein R¹ is C₁₋₁₀ alkyl or aryl, optionally respectively substituted;
B is selected from pyrrole and pyrazole, optionally respectively substituted; or
B is C;
C is selected from O—R², wherein R² is C₁₋₁₀ alkyl or aryl, optionally respectively substituted;
D is a neutral bidentate ligand, wherein said ligand is 1,10-phenanthroline or substituted 1,10-phenanthroline;
R³ and R⁴ are independently H, C₁₋₁₀ alkyl or aryl, C₁₋₁₀ alkyl and aryl being optionally substituted; and wherein only one of R³ and R⁴ is hydrogen.

2. The complex of claim 1, wherein the complex of formula I is characterized by a stability constant K with respect to the neutral ligand in a solvent, and wherein the substitution pattern of the complex of formula I in terms of at least one of D or C or D and C is selected such to adjust the stability constant K to a range of from 5 L*mol⁻¹ to 250,000 L*mol⁻¹ when measured at 298 K when the complex of formula I is dissolved in the solvent; preferably wherein the substation pattern of at least one of D or D and C is selected such to adjust the stability constant K to a range of from 5 L*mol⁻¹ to 250,000 L*mol⁻¹ when measured at 298 K when the complex of formula I is dissolved in the solvent.

3. Complex of claim 1 or 2, wherein
R¹ is C₁₋₁₀ alkyl or phenyl, respectively independently substituted with one or more of C₁₋₁₀ alkyl, C₁₋₁₀ alkoxy, phenyl, halogen, CN, and CF₃.

4. Complex of any one of the preceding claims, wherein B is pyrrole and pyrazole, respectively independently substituted with one or more of C₁₋₁₀ alkyl, C₁₋₁₀ alkoxy or phenyl.

5. Complex of any one of claims 1 to 4, wherein R² is selected from C₁₋₁₀ alkyl, independently substituted with one or more of halogen or phenyl; or is phenyl, independently substituted with one or more of C₁₋₁₀ alkyl, C₁₋₁₀ alkyl substituted with one or more of halogen, C₁₋₁₀ alkoxy, phenyl, halogen, —(CH₂)₄— to form an annulated ring with said phenyl, or —(CH═CH—CH═CH)— to form an annulated ring with said phenyl, or with —O-silyl.

6. Complex of any one of claims 1 to 5, wherein phenanthroline is independently substituted with one or more electron-donating substituents, preferably selected from C₁₋₁₀ alkyl, C₁₋₁₀ alkoxy, phenyl, or is substituted with one or more electron-withdrawing substituents, preferably selected from halogen, CN, CF₃ and CCl₃.

7. Complex of any one of claims 1 to 6, wherein R³ and R⁴ are independently H, C₁₋₁₀ alkyl or aryl, wherein C₁₋₁₀ alkyl or aryl are independently substituted with one or more of C₁₋₅ alkyl, C₁₋₅ alkyl substituted with one or more of halogen, C₁₋₅ alkoxy, phenyl, halogen.

8. Complex of any one of claims 2 to 7, wherein K is in the range of from 10 L*mol⁻¹ to 150,000 L*mol⁻¹ or 10 L*mol⁻¹ to 100,000 L*mol⁻¹ or 10 L*mol⁻¹ to 50,000 L*mol⁻¹ or 10 L*mol⁻¹ to 10,000 L*mol⁻¹ or 10 L*mol⁻¹ to 5,000 L*mol⁻¹ or 10 L*mol⁻¹ to 500 L*mol⁻¹.

9. Complex of any one of claims 2 to 8, wherein the concentration of the complex in the solvent is in the range of from 0.0001 to 0.5 M.

10. Complex of any one of claims 2 to 9, wherein 1,10-phenanthroline and substituted 1,10 phenanthroline are selected in order to adjust K.

11. Complex of any one of claims 2 to 10, wherein 1,10-phenanthroline is substituted with one or more electron-donating groups in order to increase K compared to a complex of formula I having the same substitution pattern in terms of A, B and C but in which the bidentate ligand D is unsubstituted 1,10-phenanthroline.

12. Complex of any one of claims 2 to 10, wherein 1,10-phenanthroline is substituted with one or more electron-withdrawing groups in order to decrease K compared to a complex of formula I having the same substitution pattern in terms of A, B and C but in which the bidentate ligand D is unsubstituted 1,10-phenanthroline.

13. Complex of any one of claims 2 to 12, wherein C is selected in order to adjust K, wherein C is characterized in terms of its alkoxide cone angle, provided C is an alkoxide, the alkoxide cone angle and the determination thereof being defined in the description.

14. Complex of claim 13, wherein the alkoxide cone angle is increased in order to decrease K compared to a complex having the same substitution pattern in terms of A, B and D; or wherein the alkoxide cone angle is decreased in order to increase K compared to a complex having the same substitution pattern in terms of A, B and D.

15. Complex of any one of claims 2 to 12, wherein C is selected in order to adjust K, wherein C is characterized in terms of its steric bulk, provided C is an aryloxide.

16. Complex of claim 15, wherein the steric bulk is increased in order to decrease K compared to a complex having the same substitution pattern in terms of A, B and D; or wherein the steric bulk is decreased in order to increase K compared to a complex having the same substitution pattern in terms of A, B and D.

17. Complex of any one of the preceding claims wherein the complex is of formula 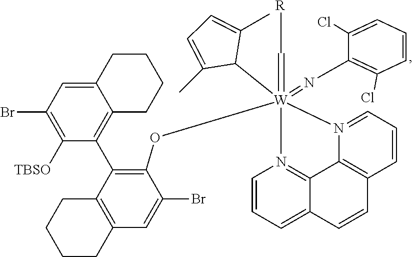
wherein R is C(CH₃)₃, C(CH₃)₂C₆H_{5,} C₆H₅, or o-(C₁₋₄)-alkoxy C₆H₄;
preferably wherein R is o-(C₁₋₄)-alkoxy C₆H₄;
more preferably wherein the complex is 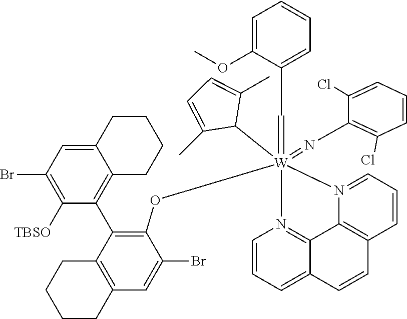
[O-TBS=O-Si(t-butyl)(Me)₂].

18. Method of making a complex as defined in any one of claims 1 to 17, comprising:
subjecting a complex of formula II 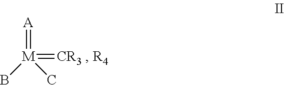
wherein A, B, C, R³ and R⁴ have the meaning as defined with respect to the complex of formula I, to
1,10-phenanthroline or substituted 1,10-phenanthroline,
in the solvent.

19. Method of claim 18, further comprising isolating the complex of formula I in solid form.

20. Method of claim 18 or 19, wherein the complex of formula I is isolated by filtration.

21. Method of claim 18 or 19, wherein the complex of formula I is isolated by evaporating the solvent.

22. System comprising
a complex of formula I as defined in any one of claims 1 to 17 and
a complex of formula II as defined in claim 18;
wherein the complex of formula I and the complex of formula II are dissolved in the solvent.

23. System of claim 22, wherein the system does not comprise a Lewis acid.

24. System of claim 23, wherein the Lewis acid is MgCl₂, MgBr₂, Mgl₂, MnCl₂, MnBr₂, MnI₂, FeCl₃, AlCl₃, CuCl₂, ZnCl₂, ZnBr₂, Znl₂, Zn(triflate)₂ or Zn(trifluoroacetate)₂.

25. Method of making a complex of formula II as defined in claim 18 comprising:
dissolving the complex of formula I as defined in any one of claims 1 to 17 in the solvent.

26. Method of claim 25, wherein the method is performed in the absence of a Lewis acid.

27. Method of claim 26, wherein the Lewis acid is MgCl₂, MgBr₂, Mgl₂, MnCl₂, MnBr₂, MnI₂, FeCl₃, AlCl₃, CuCl₂, ZnCl₂, ZnBr₂, Znl₂, Zn(triflate)₂ or Zn(trifluoroacetate)₂.

28. Method of performing a metathesis reaction of a compound comprising an olefinic double bond, comprising:
adding a complex as defined in any one of claims 1 to 17 to the olefinic compound in presence of the solvent.

29. Method of claim 28 wherein the compound comprising an olefinic double bond is the solvent.

30. Method of claim 28 or 29, wherein the metathesis reaction is performed in absence of a Lewis acid.

31. Method of claim 30, wherein the Lewis acid is MgCl₂, MgBr₂, Mgl₂, MnCl₂, MnBr₂, MnI₂, FeCl₃, AlCl₃, CuCl₂, ZnCl₂, ZnBr₂, Znl₂, Zn(triflate)₂ or Zn(trifluoroacetate)₂.
